(19) United States
(12) Patent Application Publication
(10) Pub. No.: US 2016/0362311 A1
(43) Pub. Date: Dec. 15, 2016

(54) METHOD OF TREATING LIQUID OR OBJECT USING GENERATION OF PLASMA IN OR NEAR LIQUID

(71) Applicant: Panasonic Intellectual Property Management Co., Ltd., Osaka (JP)

(72) Inventors: HIROKAZU KIMIYA, Kyoto (JP); SHIN-ICHI IMAI, Osaka (JP)

(21) Appl. No.: 15/171,766

(22) Filed: Jun. 2, 2016

(30) Foreign Application Priority Data
Jun. 9, 2015 (JP) 2015-117025

(51) Int. Cl. C02F 1/461; C02F 1/467; A61L 2/18

(52) U.S. Cl. CPC C02F 1/4618; A61L 2/18; C02F 1/467; C02F 2001/46185; C02F 2303/04

(57) ABSTRACT

The method includes: preparing a liquid having a pH of 9 or more; and generating plasma in or near the liquid to generate a plasma-treated liquid having a pH of 9 or more.

### BACKGROUND

1. Technical Field

The present disclosure relates to a method of treating a liquid, a method of treating an object, a liquid treatment apparatus, and a plasma-treated liquid.

2. Description of the Related Art

Sterilization apparatuses utilizing plasma for cleaning and sterilizing water have been known. For example, Japanese Unexamined Patent Application Publication No. 2009-255027 discloses a sterilization apparatus for sterilizing microorganisms or bacteria with active species produced in water by means of plasma.

### SUMMARY

A method according to an aspect of the present disclosure comprises: preparing a liquid having a pH of 9 or more; and generating plasma in or near the liquid to generate a plasma-treated liquid having a pH of 9 or more.

It should be noted that comprehensive or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating an example of the structure of a treatment liquid generation apparatus according to an Embodiment;

FIG. 2 is a flow chart showing an example of a method of generating a treatment liquid according to the Embodiment;

FIG. 3 is a flow chart showing the step of preparing a first treatment liquid according to the Embodiment;

FIG. 4A is a flow chart showing an example of a method of treating an object according to the Embodiment;

FIG. 4B is a flow chart showing an example of the method of treating an object according to the Embodiment;

FIG. 5 is a graph showing the results of a test of indigo carmine decomposition by the liquid samples according to Examples 1 and 3 and Comparative Example 4;

FIG. 6 is a graph showing the results of a test of indigo carmine decomposition by the liquid samples prepared by leaving the liquid samples according to Examples 1 and 3 and Comparative Example 4 to stand for 24 hours;

FIG. 7 is a graph showing the results of a test of indigo carmine decomposition by the liquid samples according to Examples 2 and 4 and Comparative Example 4;

FIG. 8 is a graph showing the results of a test of indigo carmine decomposition by the liquid samples prepared by leaving the liquid sample according to Comparative Example 1 to stand for predetermined periods of time;

FIG. 9 is a graph showing the results of a test of indigo carmine decomposition by the liquid samples according to Comparative Examples 2, 4, and 5; and

FIG. 10 is a diagram illustrating the structure of a treatment liquid generation apparatus according to a modification example of an Embodiment.

### DETAILED DESCRIPTION

### Definition of Terms

The term “alkaline” means that the pH (hydrogen ion exponent) is 9 or more; and the term “acidic” means that the pH is less than 6.

The term “alkalinization” means that the pH is adjusted to 9 or more; and the term “acidification” means that the pH is adjusted to less than 6.

The term “plasma treatment” means bringing of plasma into contact with a liquid or bringing of a gas containing active species produced by means of plasma into contact with a liquid.

The term “liquid to be plasma-treated” refers to a liquid before treatment with plasma.

The term “plasma-treated liquid” refers to a liquid after treatment with plasma. The plasma-treated liquid, for example, can function as a treatment liquid for decomposing and/or sterilizing an object. For simplification of explanation, a plasma-treated liquid before adjustment of the pH may be called a first treatment liquid, and a plasma-treated liquid after adjustment of the pH may be called a second treatment liquid.

The term “method of treating a liquid” refers to a method of treating a liquid with plasma and/or changing the pH of the liquid. When a liquid subjected to the method of treating a liquid is utilized as a treatment liquid for decomposing and/or sterilizing an object, the method of treating a liquid may be called a method of generating a treatment liquid. That is, the “method of generating a treatment liquid” is an example of the method of treating a liquid. Similarly, a “treatment liquid generation apparatus” is an example of a liquid treatment apparatus.

The term “object” refers to a material to be decomposed and/or sterilized with a plasma-treated liquid.

The term “preparing a liquid” refers to not only generating a liquid but also procuring a liquid.

The term “near a liquid” refers to a region apart from the liquid surface in an area where the active species produced by means of plasma can come into contact with liquid, for example, a region within a distance of 2 cm from the liquid surface.

The term “adding of A to B” means not only that A and B are mixed by supplying A to B but also that A and B are mixed by supplying B to A, unless specifically mentioned.

### Overview of Embodiments

A method of generating a treatment liquid according to an embodiment of the present disclosure includes: preparing a liquid having a pH of 9 or more; and generating plasma in or near the liquid to generate a treatment liquid having a pH of 9 or more.

The alkaline treatment liquid generated by this method has a high activity and excellent durability of the activity. Accordingly, the treatment liquid can be used for, for example, decomposing and/or sterilizing an object, such as an organic material, a microorganism, or a bacterium.

For example, the second treatment liquid having a pH of less than 6 may be generated by preparing the above-described treatment liquid as a first treatment liquid and adjusting the pH of the first treatment liquid.

The acidic second treatment liquid generated by this method has a high activity. Accordingly, for example, an object that is hardly decomposed and/or sterilized with an alkaline first treatment liquid can be decomposed and/or sterilized with the acidic second treatment liquid.

For example, the pH of the first treatment liquid may be adjusted by adding, to the first treatment liquid, (i) an acid or salt; (ii) a solution containing at least one of acids and salts; (iii) a gas or solid that can be dissolved in the first treatment liquid to become an acid; or (iv) a solution containing a microorganism producing the gas or the solid, to generate a second treatment liquid.

In such a case, the second treatment liquid can be readily generated. That is, the material in the generation of a second treatment liquid from a first treatment liquid can be selected from a variety of materials. Accordingly, for example, the cost can be reduced by selecting an inexpensive material.

The method of treating an object according to an embodiment of the present disclosure includes: one of the above-described methods of generating a treatment liquid; and bringing the generated treatment liquid into contact with an object.

The generated treatment liquid can efficiently decompose and/or sterilize the object. Accordingly, for example, the time necessary for decomposing and/or sterilizing microorganisms or bacteria can be shortened.

The method of treating an object according to an embodiment of the present disclosure includes: one of the above-described methods of generating a treatment liquid; bringing the liquid into contact with an object; and generating plasma in or near the liquid in the state that the liquid and the object are in contact with each other to generate the treatment liquid having a pH of 9 or more.

In such a case, the treatment liquid can be brought into contact with an object while being generated. Accordingly, the generated treatment liquid can efficiently decompose and/or sterilize the object, and, for example, the time necessary for decomposing and/or sterilizing microorganisms or bacteria can be shortened.

The treatment liquid according to an embodiment of the present disclosure is generated by the method of generating a treatment liquid.

This treatment liquid can efficiently decompose and/or sterilize the object. Accordingly, for example, the time necessary for decomposing and/or sterilizing microorganisms or bacteria can be shortened.

The treatment liquid generation apparatus according to an embodiment of the present disclosure includes: a container for containing a liquid; a plasma generator including at least one electrode pair and a power supply for applying a voltage to the electrode pair to generate plasma in or near the liquid in the container; and a control circuit for controlling the plasma generator. The control circuit starts generation of plasma by the plasma generator, and stops the generation of plasma when the average pH per unit time of the liquid in the container is within a predetermined range of 9 or more.

The alkaline treatment liquid after the generation of plasma contains active species, such as ions, molecules, and radicals, has a high activity, and excellent durability of the activity. Accordingly, for example, the treatment liquid can be used for decomposition and/or sterilization of an object, such as an organic material, a microorganism, or a bacterium.

The treatment liquid generation apparatus according to an embodiment of the present disclosure includes a container for containing a liquid, a feeder for supplying a pH regulator to the container for adjusting the pH of the liquid in the container, and a control circuit for controlling the feeder. When the container contains a first treatment liquid having a pH of 9 or more generated by generating plasma in or near the liquid, the control circuit instructs the feeder to supply the pH regulator to the container so as to adjust the pH of the first treatment liquid in the container to less than 6. A second treatment liquid may be thus generated.

The generated acidic second treatment liquid contains active species, such as ions, molecules, and radicals, and therefore has a high activity, and excellent durability of the activity. Accordingly, for example, an object that is hardly decomposed and/or sterilized with an alkaline first treatment liquid can be decomposed and/or sterilized with the acidic second treatment liquid.

Embodiments will now be specifically described with reference to the drawings.

Incidentally, the embodiments described below all show comprehensive or specific examples. The numbers, shapes, materials, components, the arrangement configuration and connection configuration of the components, steps, the order of the steps, etc. shown in the following embodiments are merely examples and are not intended to limit the present disclosure. Among the components in the following embodiments, components that are not mentioned in any independent claim describing the broadest concept will be described as optional components. In the embodiments, the method of generating a treatment liquid will be described as an example of operation of the treatment liquid generation apparatus, but is not limited to a specific apparatus structure.

### EMBODIMENT

### 1. Treatment Liquid Generation Apparatus

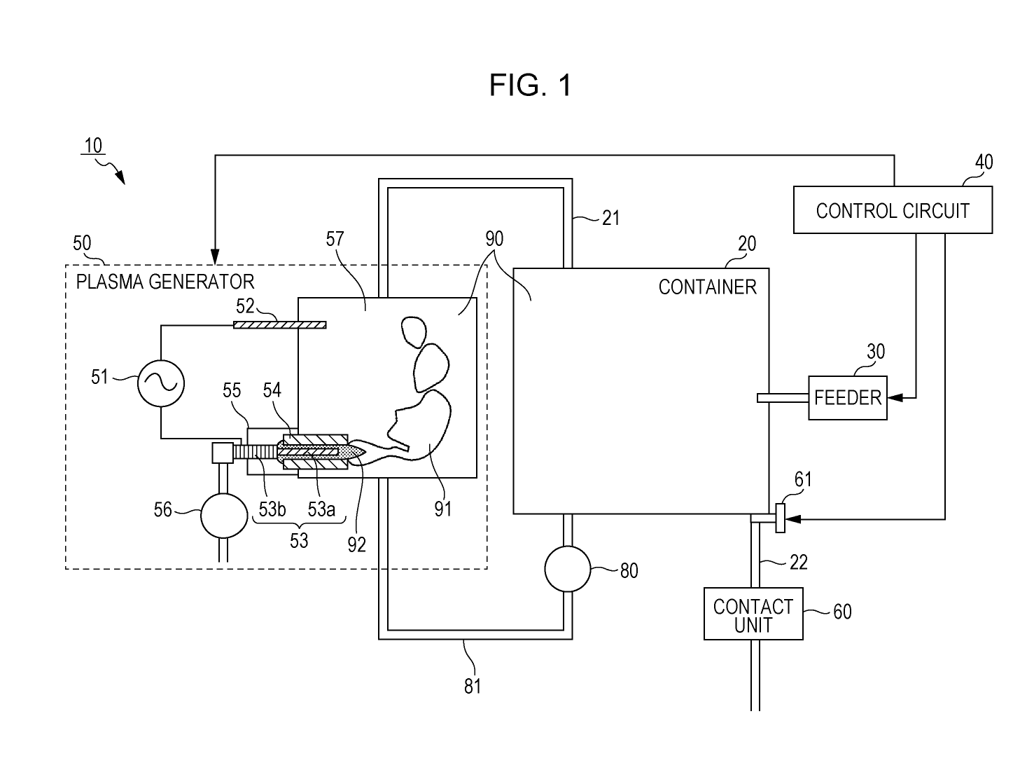

The outline of the treatment liquid generation apparatus according to an Embodiment will be described referring to FIG. 1. FIG. 1 shows an example of the structure of a treatment liquid generation apparatus 10 according to the Embodiment.

The treatment liquid generation apparatus 10 generates plasma in or near an alkaline liquid to generate an alkaline first treatment liquid. As shown in FIG. 1, the treatment liquid generation apparatus 10 includes a container 20, a control circuit 40, a plasma generator 50, a circulation pump 80, and a pipe 81.

The treatment liquid generation apparatus 10 may further adjust the pH of the first treatment liquid to generate an acidic second treatment liquid. For example, as shown in FIG. 1, the treatment liquid generation apparatus 10 further includes a feeder 30 for supplying a pH regulator to the container 20.

The treatment liquid generation apparatus 10 may further brining an alkaline first treatment liquid or acidic second treatment liquid into contact with an object. For example, as shown in FIG. 1, the treatment liquid generation apparatus 10 further includes a contact unit 60 for bringing a treatment liquid into contact with an object and a valve 61.

Examples of the components of the treatment liquid generation apparatus 10 according to the Embodiment will now be described in detail.

### 1-1. Container

The container 20 is for containing a liquid. The container 20 is provided with an inlet 21 and an outlet 22.

The container 20 is made of, for example, a material resistant to acid or alkali. For example, the container 20 is formed from a resin material, such as polyvinyl chloride or tetrafluoroethylene (PFA), a metal material, such as stainless steel, or a ceramic. The container 20 may have any size and any shape.

The container 20 may contain a liquid 90 having a pH of 9 or more as a liquid to be plasma-treated. As shown in FIG. 1, the container 20 is connected to the plasma generator 50. The plasma generator 50 subjects the liquid 90 to plasma treatment. For example, the plasma generator 50 generates plasma in or near the liquid 90 to generate a first treatment liquid having a pH of 9 or more. As a result, the first treatment liquid may be contained in the container 20.

The liquid 90 is, for example, a buffer solution, such as a phosphate buffer solution, or an aqueous sodium hydroxide solution. When the liquid 90 is a buffer solution, the pH can be gently changed and can be readily adjusted to a desired level.

### 1-2. Feeder

The feeder 30 supplies, to the container 20, a pH regulator for adjusting the pH of the liquid 90 in the container 20. The feeder 30 supplies, for example, a predetermined amount of a pH regulator to the container 20 with a predetermined timing on the basis of the instruction from the control circuit 40. The feeder 30 adds, for example, a solution containing an acid or a base as a pH regulator to the alkaline first treatment liquid to adjust the pH of the treatment liquid to less than 6.

The pH regulator is (i) an acid or a base; (ii) a solution containing at least one acid or base; (iii) a gas or solid that can be dissolved in a liquid to become an acid; or (iv) a solution containing a microorganism producing the gas or the solid. For example, the pH regulator is sulfuric acid (H₂SO₄), nitric acid (HNO₃), or a salt such as aluminum sulfate (Al₂(SO₄)₃) or magnesium chloride (MgCl₂). These pH regulators are merely examples, and the pH regulator may be in any form, such as a solid, liquid, or gas, as long as the material can adjust the pH of a liquid. Alternatively, the pH regulator may be a microorganism that produces a material capable of adjusting the pH.

The feeder 30 may add a solution containing a base as a pH regulator for preventing the pH of the liquid 90 from decreasing to less than 9 during the plasma treatment. In this case, the pH regulator may be (i) a base; (ii) a solution containing a base; (iii) a gas or solid that can be dissolved in a liquid to become a base; or (iv) a solution containing a microorganism producing the gas or the solid. For example, the pH regulator may be an aqueous sodium hydroxide (NaOH) solution or an aqueous ammonia (NH₃) solution.

The feeder 30 includes, for example, a container for containing a pH regulator, a pump, and a valve, connected to the container, for supplying the pH regulator to the container 20. For example, the control circuit 40 controls the pump to regulate the pressure difference between the container containing the pH regulator and the container 20 containing a liquid 90. For example, the control circuit 40 controls the switching operation of the valve.

### 1-3. Control Circuit

The control circuit 40 controls the plasma generator 50.

The control circuit 40 controls, for example, the power supply 51 of the plasma generator 50 and the gas feeder 56. The control circuit 40 controls the timing and the period of applying a voltage by the power supply 51 between the first electrode 52 and the second electrode 53. That is, the control circuit 40 controls the timing of generating plasma 92 in the liquid 90 and the period of the plasma generation (i.e., the duration of the plasma treatment). In addition, the control circuit 40 controls, for example, the timing and the amount of the gas supply to the liquid 90 by the gas feeder 56.

For example, the control circuit 40 places an alkaline liquid to be plasma-treated in the container 20 and then instructs the plasma generator 50 to start generation of plasma 92 and to stop the generation of plasma 92 after the elapse of a predetermined time. For example, the control circuit 40 includes a timer for measuring the elapsed time from the time of starting the plasma treatment. The control circuit 40 stops the generation of plasma when the duration of the plasma treatment reached to a predetermined time.

The control circuit 40 may further control the feeder 30. For example, when the container 20 contains an alkaline first treatment liquid, the control circuit 40 may instruct the feeder 30 to supply a pH regulator to the container 20 to adjust the pH of the first treatment liquid in the container 20. As a result, an acidic second treatment liquid is generated. That is, the second treatment liquid is an acidic plasma-treated liquid generated by adjusting the pH of the alkaline plasma-treated liquid. The second treatment liquid may be discharged to the outside from the outlet 22 of the container 20, as necessary, and may be used for, for example, decomposition and/or sterilization of an object.

The control circuit 40 may instruct the feeder 30 to supply a pH regulator to the container 20 during the generation of plasma by the plasma generator 50. For example, when the gas feeder 56 in the plasma generator 50 supplies air, a part of nitrogen in the air can be oxidized into nitric acid to be dissolved in the liquid 90 to reduce the pH of the liquid 90. Accordingly, the control circuit 40 can prevent the pH of the liquid from decreasing to be less than 9 by supplying, for example, a solution containing a base, as a pH regulator, to the container 20.

Alternatively, the control circuit 40 may instruct the plasma generator 50 to stop the generation of plasma 92 and then instruct the feeder 30 to supply a pH regulator to the container 20. As a result, an alkaline first treatment liquid is generated in the container 20.

Alternatively, the control circuit 40 may successively adjust the pH such that the average pH per unit time of the liquid 90 in the container 20 is 9 or more during the generation of plasma 92 by the plasma generator 50. As a result, the alkaline first treatment liquid is generated in the container 20.

The control circuit 40 includes, for example, a non-volatile memory storing a program and a processor executing the program. The control circuit 40 may further include a volatile memory, which is a temporary storage area for executing the program, and input and output ports. The control circuit 40 is, for example, a microcomputer.

### 1-4. Plasma Generator

The plasma generator 50 generates plasma 92 in a liquid 90. For example, the plasma generator 50 shown in FIG. 1 generates plasma 92 in a bubble 91 formed in the liquid 90. The bubble 91 is, for example, formed from the gas supplied by the gas feeder 56.

As shown in FIG. 1, the plasma generator 50 includes a power supply 51, a first electrode 52, a second electrode 53, an insulator 54, a holding block 55, a gas feeder 56, and a reaction tank 57. Examples of each component of the plasma generator 50 will now be described in detail.

The power supply 51 is connected between the first electrode 52 and the second electrode 53. The power supply 51 supplies a predetermined voltage between the first electrode 52 and the second electrode 53. The predetermined voltage is, for example, a pulse voltage or an AC voltage. The predetermined voltage is, for example, 1 to 50 kV with a voltage pulse of 1 to 100 kHz. The voltage waveform may be, for example, any of pulse, half sine, and sine waveforms. The value of the current flowing between the first electrode 52 and the second electrode 53 is, for example, 1 mA to 3 A. For example, the power supply 51 applies, between the first electrode 52 and the second electrode 53, a pulse voltage having a peak voltage of 4 kV, a pulse width of 1 μsec, and a frequency of 30 kHz. For example, the input power by the power supply 51 is 10 to 100 W.

The first electrode 52, one of an electrode pair, is disposed so as to pass through the wall of the reaction tank 57. The first electrode 52 is at least partially in contact with the liquid 90. The first electrode 52 is, for example, a rod-like electrode. The first electrode 52 is, for example, made of a conductive metal material, such as copper, aluminum, or iron.

The second electrode 53, the other of the electrode pair, is disposed so as to pass through the wall of the reaction tank 57. The second electrode 53 is at least partially in contact with the liquid 90, at least when no power is supplied from the power supply 51. The second electrode 53 is used as a reaction electrode. When a predetermined voltage is applied between the first electrode 52 and the second electrode 53, plasma 92 is generated in the circumference of the second electrode 53. For example, the plasma 92 is generated in the bubble 91.

In the example shown in FIG. 1, the second electrode 53 includes a metal electrode portion 53a and a metal screw portion 53b.

The metal electrode portion 53a is press-inserted into the metal screw portion 53b and is unified to the metal screw portion 53b. The metal electrode portion 53a is formed so as not to protrude from the opening of the insulator 54. The metal electrode portion 53a is, for example, a rod-like electrode and is formed from a plasma-resistant metal material, such as tungsten. Alternatively, though the durability is decreased, the metal electrode portion 53a may be formed from, for example, copper, aluminum, or iron.

The metal screw portion 53b supports the press-inserted metal electrode portion 53a. The metal screw portion 53b is, for example, a rod-like member and is formed from iron. Alternatively, the metal screw portion 53b may be made of, for example, copper, zinc, aluminum, tin, or brass, instead of iron.

The metal screw portion 53b includes a screw part (e.g., male screw) that is screwed into a screw part (e.g., female screw) provided to the holding block 55. Such a structure can adjust the positional relation between the metal electrode portion 53a and the insulator 54.

The metal screw portion 53b is, for example, provided with a through-hole (not shown) passing through in the axial direction. One end of the through-hole communicates with the gap between the metal electrode portion 53a and the insulator 54. The other end of the through-hole is connected to the gas feeder 56. Accordingly, the gas supplied from the gas feeder 56 is supplied to the liquid 90 through the through-hole and the gap and thereby forms a bubble 91 in the liquid 90.

The insulator 54 is disposed so as to surround the outer surface of the metal electrode portion 53a. The insulator 54 has, for example, a cylindrical shape. The insulator 54 has an inner diameter larger than the outer diameter of the metal electrode portion 53a. Consequently, a gap is formed between the inner surface of the insulator 54 and the outer surface of the metal electrode portion 53a.

The insulator 54 may be formed from, for example, an alumina ceramic or may be formed, for example, magnesia, quartz, or yttrium oxide.

The holding block 55 is a member for supporting the metal screw portion 53b and the insulator 54. The holding block 55 is provided with a screw part (e.g., female screw). The positional relation between the holding block 55 and the metal screw portion 53b can be controlled by rotating the metal screw portion 53b around the axis. Such a structure can adjust the positional relation between the insulator 54 and the metal electrode portion 53a. For example, the front edge of the metal electrode portion 53a can be adjusted not to protrude from the opening of the insulator 54.

The gas feeder 56 supplies a gas to the liquid 90, and thereby a bubble 91 is formed in the liquid 90. The bubble 91 is discharged into the liquid 90 in the reaction tank 57 through the opening of the insulator 54. The gas feeder 56 is, for example, a pump.

The gas feeder 56 takes in, for example, the air present in the periphery of the plasma generator 50 and then supplies this air to the liquid 90 in the reaction tank 57. The gas supplied by the gas feeder 56 is not limited to air and may be any gas that can be ionized into a plasma form, such as nitrogen, oxygen, a noble gas, such as argon, or water vapor. The gas is supplied to the liquid 90 through the through-hole provided to the metal screw portion 53b and the gap between the metal electrode portion 53a and the insulator 54, and thereby the gas forms a bubble 91 in the liquid 90. The metal electrode portion 53a is, for example, covered with the bubble 91 and can be kept in a state of not being in direct contact with the liquid 90. In this state, plasma 92 can be generated in the bubble 91.

The reaction tank 57 is a container for generating plasma 92 therein. The reaction tank 57 is connected to the pipe 81. The circulation pump 80 circulates the liquid 90 between the reaction tank 57 and the container 20 through the pipe 81. The reaction tank 57 may be a part of the pipe 81.

For example, the circulation pump 80 sends the liquid 90 from the container 20 to the reaction tank 57, within which plasma 92 is generated in the liquid 90 to thereby generate a first treatment liquid. The first treatment liquid generated in the reaction tank 57 is supplied to the container 20 through the inlet 21.

The reaction tank 57 is formed from, for example, a material resistant to acid and/or alkali. For example, the reaction tank 57 is formed from a resin material, such as polyvinyl chloride or tetrafluoroethylene (PFA), a metal material, such as stainless steel, or a ceramic. The reaction tank 57 may have any size and any shape.

The reaction tank 57 and the container 20 may be unified. That is, the plasma generator 50 may not have the reaction tank 57 and may generate plasma 92 in the container 20. In such a case, the treatment liquid generation apparatus 10 may not have the circulation pump 80 and the pipe 81.

The treatment liquid generation apparatus 10 according to the Embodiment may not have the plasma generator 50. In such a case, for example, a first treatment liquid generated in advance at another place is placed in the container 20.

### 1-5. Contact Unit and Valve

The contact unit 60 is a portion for bringing the first treatment liquid or the second treatment liquid into contact with an object. The contact unit 60 is connected to, for example, the outlet 22 of the container 20 through the valve 61. The contact unit 60 may be, for example, a container for containing an object. In such a case, the first or second treatment liquid is placed in the container through the outlet 22 to bring the first treatment liquid or the second treatment liquid into contact with the object. Alternatively, the contact unit 60 may be, for example, an injector, a spray, or a diffuser. In such a case, the first treatment liquid or the second treatment liquid is sprayed toward the object to be brought into contact with the object.

The object is a material to be decomposed and/or sterilized by the first treatment liquid or the second treatment liquid. The object is, for example, an organic material, a microorganism, or a bacterium. The contact unit 60 brings the first treatment liquid or the second treatment liquid discharged from the outlet 22 into contact with, for example, a material containing an object. The material containing an object is, for example, daily commodities, such as tableware, medical instrument, or a building material, such as the floor or window glass of a bathroom. Alternatively, the material containing an object is, for example, the human oral cavity containing a pathogen of dental caries or periodental disease; or a food, animal, or a plant containing putrefactive bacteria.

The valve 61 is provided to the outlet 22, and the switching thereof is controlled by the control circuit 40. For example, the liquid contained in the container 20 is supplied to the contact unit 60 through the outlet 22 by opening the valve 61 and is then brought into contact with an object. For example, after the generation of a second treatment liquid, the control circuit 40 opens the valve 61 to bring the second treatment liquid into contact with the object. Alternatively, the control circuit 40 brings the first treatment liquid into contact with an object by opening the valve 61 without controlling the feeder 30.

The treatment liquid generation apparatus 10 may bring the first treatment liquid or the second treatment liquid into contact with an object by means other than the contact unit 60.

For example, the treatment liquid generation apparatus 10 may further include a feeder (not shown) for supplying an object to the container 20. The feeder may be an inlet provided to the container 20 for supplying an object to the container 20 by a user. The feeder may further include a container for containing an object, and the container may be connected to the inlet through a valve. In such a structure, for example, the feeder supplies the object to the container 20 to form a mixture of the object and the first treatment liquid, and the pH of the first treatment liquid (or the mixture of the first treatment liquid and the object) can be then adjusted. In such a case, generation of a second treatment liquid and contact of the second treatment liquid with the object can be concurrently performed.

For example, the treatment liquid generation apparatus 10 may include a container for containing a mixture of an object and a pH regulator. In such a structure, the mixture may be brought into contact with a first treatment liquid by supplying the mixture to the first treatment liquid or by supplying the first treatment liquid to the mixture. In both cases, the pH of the first treatment liquid is adjusted concurrently with the contact of the first treatment liquid with the object. As a result, generation of a second treatment liquid and contact of the second treatment liquid with the object can be concurrently performed. Alternatively, for example, an object and a pH regulator may be concurrently supplied to the container 20 from different containers.

### 1-6. Circulation Pump and Pipe

The circulation pump 80 is an example of the liquid feeder provided to the pipe 81. The circulation pump 80 is, for example, a chemical pump.

The circulation pump 80 circulates the liquid 90 between the container 20 and the reaction tank 57 through the pipe 81. That is, the circulation path of the liquid 90 is composed of the container 20, the pipe 81, and the reaction tank 57.

The pipe 81 is a tube for forming the circulation path for circulating the liquid 90. The pipe 81 is formed from, for example, a tubular member, such as a pipe, tube, or hose. The pipe 81 is formed from, for example, the same material as that of the container 20.

### 2. Operation

### 2-1. Method of Generating Treatment Liquid

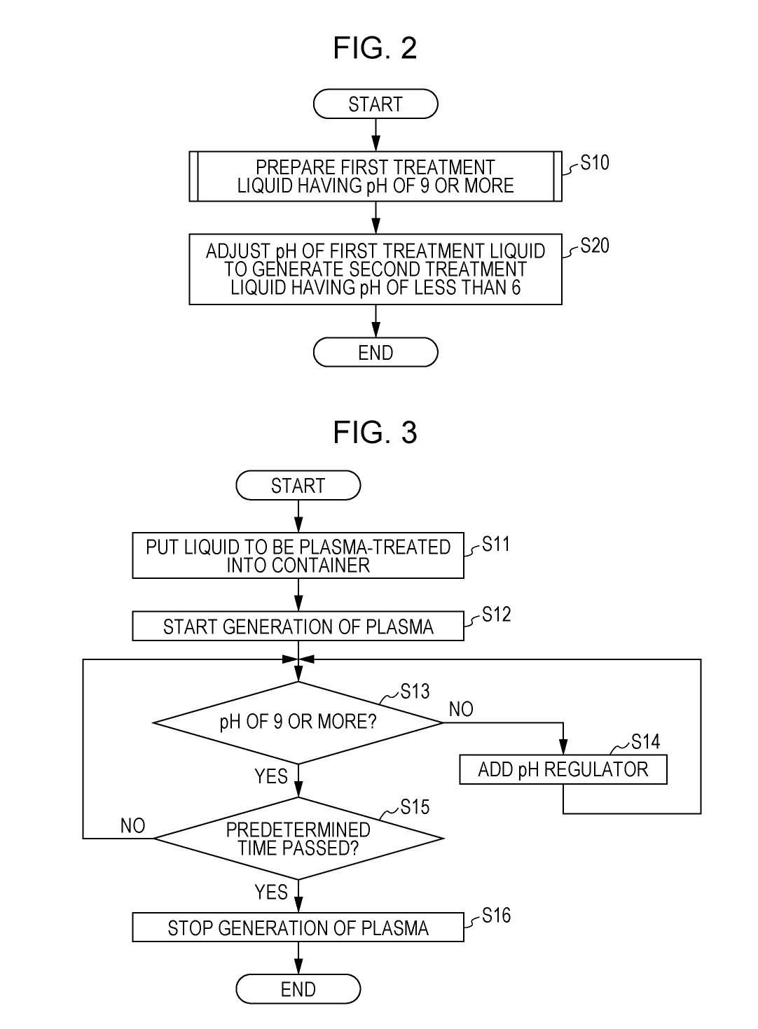

Examples of the operation of the treatment liquid generation apparatus 10 according to the Embodiment will be described using FIGS. 2 to 4B. A method of generating a treatment liquid according to the Embodiment will be described using FIG. 2.

FIG. 2 is a flow chart showing a method of generating a treatment liquid according to the Embodiment.

First, a first treatment liquid having a pH of 9 or more is prepared (S10). The prepared first treatment liquid is contained in the container 20.

Subsequently, the treatment liquid generation apparatus 10 adjusts the pH of the first treatment liquid to generate a second treatment liquid having a pH of less than 6 (S20). For example, the feeder 30 adds a solution containing an acid or salt to the first treatment liquid based on the instruction from the control circuit 40.

In this Embodiment, the preparation (e.g., generation) of the first treatment liquid (S10) and the generation of the second treatment liquid (S20) are performed by different procedures. For example, the first treatment liquid is generated by plasma treatment, and the second treatment liquid is generated by adding a pH regulator to the first treatment liquid. In the generation of the second treatment liquid from the first treatment liquid, plasma treatment is not performed.

For example, the first treatment liquid is stored in a container for storage. The first treatment liquid may be discharged from the storage container and then be supplied to a reaction container. An amount of the first treatment liquid which is supplied to the reaction container may be determined based on the input from a user. A pH regulator is added to the first treatment liquid in the reaction container to generate a second treatment liquid. As a result, the generated second treatment liquid can be used for decomposition and/or sterilization of the object.

### 2-2. Generation of First Treatment Liquid

A step of generating an alkaline first treatment liquid will be described using FIG. 3 as an example of the step of preparing an alkaline first treatment according to the Embodiment. FIG. 3 is a flow chart showing the step of preparing a first treatment liquid according to the Embodiment.

First, a liquid to be plasma-treated is placed in the container 20 (S11). The liquid to be plasma-treated is a liquid 90 not subjected to the plasma treatment and is, for example, an alkaline buffer solution or an aqueous solution.

Subsequently, generation of plasma is started (S12). For example, the gas feeder 56 supplies a gas to the liquid 90 based on the instruction from the control circuit 40. The second electrode 53 is covered with the bubble 91 of the supplied gas. In this state, the power supply 51 applies a voltage between the first electrode 52 and the second electrode 53 based on the instruction from the control circuit 40. As a result, electric discharge is caused in the bubble 91 to generate plasma 92 therein. The generated plasma 92 acts on the liquid 90 and changes the ionic composition of the liquid 90 to vary the pH of the liquid 90. Alternatively, the plasma 92 acts on the supplied gas to generate a product, and the product is dissolved in the liquid 90 to vary the pH of the liquid 90.

For example, when the gas feeder 56 supplies air to the liquid 90, a part of nitrogen in the supplied air is oxidized to nitric acid by the plasma 92. This nitric acid is dissolved in the liquid 90 to reduce the pH of the liquid 90. Accordingly, in the flow chart shown in FIG. 3, when the average pH per unit time of the liquid 90 is less than 9 (the case of “No” in S13), the control circuit 40 instructs the feeder 30 to supply a pH regulator to the liquid 90 in the container 20 (S14). The pH regulator on this occasion is a material for increasing the pH of the liquid 90, such as a solution containing a base.

When the pH is 9 or more (the case of “Yes” in S13) and when the predetermined period of time has passed (the case of “Yes” in S15), the generation of plasma 92 is stopped (S16). For example, the control circuit 40 instructs the power supply 51 to stop the application of a voltage between the first electrode 52 and the second electrode 53. The control circuit 40 also instructs the gas feeder 56 to stop the supply of a gas.

The predetermined period of time is a time for continuing plasma treatment and is arbitrarily determined. The predetermined period of time is, for example, a sufficient time for the first treatment liquid (or the second treatment liquid) to gain desired decomposition and/or sterilization ability. The decomposition and/or sterilization ability of the first treatment liquid (or the second treatment liquid) is enhanced with an increase in the period of time for generating plasma 92 (i.e., the duration of plasma treatment).

The container 20 may be provided with a pH sensor for detecting the pH of the liquid 90. The control circuit 40 may receive the pH value of the liquid 90 from the pH sensor and may stop the generation of plasma 92 based on the received pH value.

The pH sensor is, for example, a glass electrode pH meter. The glass electrode pH meter uses, for example, a potassium chloride solution or an ionic liquid salt bridge as a liquid junction, and uses Ag/AgCl as electrodes. The pH sensor may be, for example, an ISFET pH meter. Alternatively, the determination of the pH may be colorimetric measurement including sampling a liquid and using a pH indicator or a pH test paper.

The pH sensor may not be provided. In such a case, the duration of the plasma treatment may be set to an appropriate period for maintaining the pH of the liquid 90 to 9 or more based on, for example, the type of the gas to be supplied by the gas feeder 56, the type and the volume of the liquid 90, and the voltage to be applied.

As described above, the method of generating a treatment liquid according to the Embodiment can generate a second treatment liquid having high decomposition and/or sterilization ability.

### 2-3. Method of Treating Object

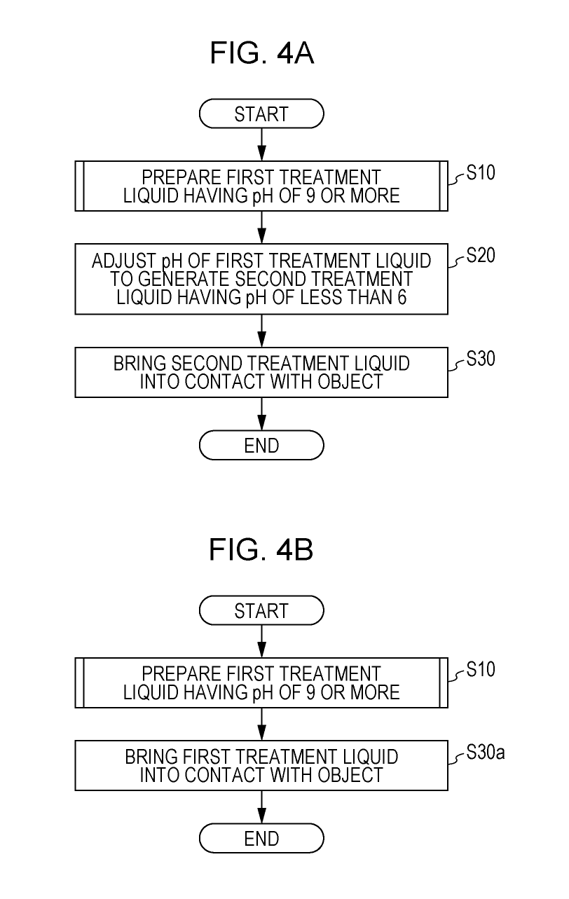

A method utilizing an acidic second treatment liquid for treating an object will now be described using FIGS. 4A and 4B.

FIG. 4A is a flow chart showing a method of treating an object according to the Embodiment. As shown in FIG. 4A, the steps, S10 and S20, until the generation of an acidic second treatment liquid are respectively the same as steps S10 and S20 shown in FIG. 2, for example.

The treatment liquid generation apparatus 10 generates a second treatment liquid and then brings the generated second treatment liquid into contact with an object (S30). For example, the control circuit 40 opens the valve 61 and thereby supplies the second treatment liquid from the container 20 to the contact unit 60 through the outlet 22. The contact unit 60 brings the supplied second treatment liquid into contact with the object.

Steps S10 and S20 may be concurrently performed. For example, the object may be mixed with a pH regulator in advance. In such a case, a mixture of the object and the pH regulator is further mixed with a first treatment liquid. Alternatively, the object, the pH regulator, and the first treatment liquid may be simultaneously mixed. That is, the method of treating an object according to the Embodiment may adjusting the pH of the first treatment liquid while allowing the first treatment liquid to be in contact with the object. In such a case, the contact of the second treatment liquid with the object can be performed concurrently with the generation of the second treatment liquid.

In the examples described above, an acidic second treatment liquid is brought into contact with an object. However, the treatment liquid is not limited thereto, and an alkaline first treatment liquid may be brought into contact with an object.

FIG. 4B is a flow chart showing the method of treating an object according to the Embodiment. As shown in FIG. 4B, a first treatment liquid is prepared in step S10, and the first treatment liquid is then brought into contact with an object (S30a).

For example, the control circuit 40 opens the valve 61 to supply the first treatment liquid from the container 20 to the contact unit 60 through the outlet 22, without supplying a pH regulator for acidifying the liquid 90 to the feeder 30. The contact unit 60 brings the supplied first treatment liquid into contact with an object.

The first treatment liquid can decompose or sterilize the object and can therefore be used, for example, for sterilization.

In the examples described above, the first treatment liquid is prepared or generated and is then brought into contact with an object, but the procedure is not limited thereto. The first treatment liquid may be brought into contact with an object while being generated. For example, a first treatment liquid having a pH of 9 or more may be generated by generating plasma in or near the liquid 90 in a state that the liquid 90 is in contact with an object.

### 3. Examples

Examples of the treatment liquid generation apparatus 10 according to the Embodiment will now be described using drawings. The present inventors prepared the following liquid samples according to Examples 1 to 4 and Comparative Examples 1 to 5 and performed a test of indigo carmine decomposition by these liquid samples for verifying the decomposition ability and the durability of the ability of first treatment liquids and second treatment liquids.

### 3-1. Conditions

The conditions of each Example and Comparative Example will now be described in detail using Tables 1 and 2. Table 1 summarizes the conditions of Examples 1 and 2 and Comparative Examples 1 to 3. Table 2 summarizes the conditions of Examples 3 and 4 and Comparative Examples 4 and 5.

TABLE 1

| | | | Comparative | Comparative | Comparative |
|---|---|---|---|---|---|
| | Example 1 | Example 2 | Example 1 | Example 2 | Example 3 |
| Material | Phosphate | NaOH solution | Standard | Phosphate | NaOH solution |
| | buffer solution | | solution | buffer solution | |
| pH | pH 12 | pH 12 | pH 6 | pH 11.5 | pH 11.5 |
| Plasma | Electric | Electric | Electric | — | — |
| treatment | discharge in a | discharge in a | discharge in a | | |
| | bubble in liquid | bubble in liquid | bubble in liquid | | |
| pH after | pH 11.4 | pH 11.2 | pH 2.5 | — | — |
| plasma | | | | | |
| treatment | | | | | |

TABLE 2

| | | | Comparative | Comparative |
|---|---|---|---|---|
| | Example 3 | Example 4 | Example 4 | Example 5 |
| Material | Phosphate buffer | NaOH solution | Phosphate buffer | Phosphate |
| | solution | | solution | buffer solution |
| pH | pH 12 | pH 12 | pH 7.2 | pH 7.2 |
| Plasma treatment | Electric discharge in | Electric discharge | — | — |
| | a bubble in liquid | in a bubble in liquid | | |
| pH after plasma | pH 11.4 | pH 11.2 | — | — |
| treatment | | | | |
| pH adjustment | Addition of sulfuric | Addition of sulfuric | Addition of | — |
| procedure | acid | acid | sulfuric acid | |
| pH after pH | pH 2.58 | pH 2.29 | pH 2.5 | — |
| adjustment | | | | |

In Examples 1 to 4 and Comparative Example 1, the liquids to be plasma-treated were plasma-treated with the treatment liquid generation apparatus 10 shown in FIG. 1. The container 20 is made of PFA and contained 100 mL of a liquid 90. The container 20 was provided with a pH sensor, and the pH and the temperature of the liquid 90 were monitored at all times.

The circulation pump 80 was a chemical pump, and the flow rate in the pipe 81 was adjusted to 0.6 L/min. The gas feeder 56 supplied air at 0.3 L/min to the liquid 90. The power supply 51 supplied a power of 20 W for 30 minutes. That is, the time for generating plasma 92, i.e., the duration of plasma treatment, was 30 minutes.

The pH of the liquid 90 is decreased when plasma 92 is generated. In order to prevent a reduction in pH to less than 9, plasma 92 was generated while, for example, an aqueous sodium hydroxide solution being appropriately added.

In Example 1, a phosphate buffer solution (i.e., liquid to be plasma-treated) having a pH of 12 was plasma-treated to prepare a liquid (i.e., first treatment liquid) having a pH of 11.4. In Example 3, sulfuric acid was added to the first treatment liquid according to Example 1 to prepare a second treatment liquid having a pH of 2.58.

In Example 2, an aqueous sodium hydroxide solution (i.e., liquid to be plasma-treated) having a pH of 12 was plasma-treated to prepare a liquid (i.e., first treatment liquid) having a pH of 11.2. In Example 4, sulfuric acid was added to the first treatment liquid according to Example 2 to prepare a second treatment liquid having a pH of 2.29.

In Comparative Example 1, a standard solution (i.e., liquid to be plasma-treated) having a pH of 6 was plasma-treated to prepare a liquid (i.e., plasma-treated liquid) having a pH of 2.5. Herein, the standard solution was an aqueous sodium sulfate (Na₂SO₄) solution adjusted so as to have a conductivity of 20 mS/m, which was equivalent to that of tap water. The standard solution was prepared by diluting 61.3 mg of sodium sulfate with ultra-pure water to 500 mL in a measuring cylinder.

In Comparative Example 2, a phosphate buffer solution (i.e., plasma-untreated liquid) having a pH of 11.5 was prepared. In Comparative Example 4, sulfuric acid was added to a phosphate buffer solution having a pH of 7.2 to prepare a phosphate buffer solution (i.e., plasma-untreated liquid) having a pH of 2.5. In Comparative Example 5, a phosphate buffer solution (i.e., plasma-untreated liquid) having a pH of 7.2 was prepared. In Comparative Examples 2, 4, and 5, the phosphate buffer solutions were not plasma-treated.

In Comparative Example 3, an aqueous sodium hydroxide solution having a pH of 11.5 was prepared. In Comparative Example 3, the aqueous sodium hydroxide solution was not plasma-treated.

The first treatment liquids in Examples 1 and 2, the second treatment liquids in Examples 3 and 4, the plasma-treated liquid in Comparative Example 1, and plasma-untreated liquids in Comparative Examples 2 to 5 were used as liquid samples of the decomposition test below.

### 3-2. Results of Decomposition Test

The results of a test of indigo carmine decomposition by the samples according to Examples 1 to 4 and Comparative Examples 1 to 5 will now be described. Indigo carmine has a light absorption maximum at a wavelength of 610 nm. If a liquid sample contains indigo carmine, light having the wavelength of 610 nm is strongly absorbed by the indigo carmine. In contrast, if the indigo carmine contained in a liquid sample is decomposed, light having the wavelength of 610 nm is hardly absorbed. Accordingly, the change with time in absorbance, when a liquid sample and indigo carmine are mixed, can be used as an index of the decomposition ability of the liquid sample.

Accordingly, the changes with time in absorbance for light having the wavelength of 610 nm in various liquid samples containing indigo carmine were measured with a spectrometer. The measurement was performed by the following two processes.

In a first measuring process, 11 μL of ultra-pure water containing 2000 ppm of indigo carmine was dropped on a glass cell for spectrophotometer, and 2.2 mL of a liquid sample having a pH adjusted to a desired level was added thereto. Immediately, pipetting was performed to start the measurement of absorbance. That is, the initial concentration of indigo carmine in this measuring process is 10 ppm.

In a second measuring process, the pH is adjusted after the start of absorbance measurement. That is, the measurement of absorbance of the first treatment liquid was started in accordance with the first measuring process, and a pH regulator was then added to the first treatment liquid to generate a second treatment liquid. As a result, the decomposition of indigo carmine by the generated second treatment liquid can be precisely measured. The second measuring process is suitable when the ability of decomposing indigo carmine is high.

In the experiments described below, Examples 2 and 4 employed the second measuring process, and other Examples employed the first measuring process.

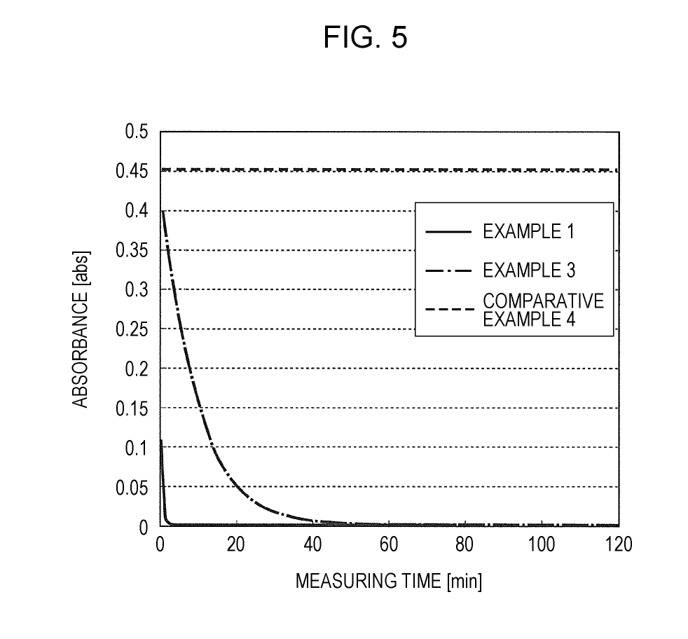

FIG. 5 shows the results of a test of indigo carmine decomposition by the liquid samples of Examples 1 and 3 and Comparative Example 2.

As shown in FIG. 5, in Example 1, the absorbance sharply decreased immediately after the contact of the liquid sample to indigo carmine. That is, the plasma-treated alkaline phosphate buffer solution promptly decomposed indigo carmine.

In Example 3, the absorbance sharply decreased immediately after the contact of the liquid sample to indigo carmine. That is, the phosphate buffer acidified after plasma treatment sufficiently decomposed indigo carmine. The time necessary for decomposing indigo carmine in Example 1 was shorter than that in Example 3. That is, the first treatment liquid according to Example 1 had a higher decomposition ability than the second treatment liquid according to Example 3.

In contrast, in Comparative Example 4, the absorbance did not substantially change. That is, the acidified plasma-untreated liquid did not substantially decompose indigo carmine. The comparison of Example 3 and Comparative Example 4 demonstrates that plasma treatment contributes to expression of decomposition ability of the treatment liquid.

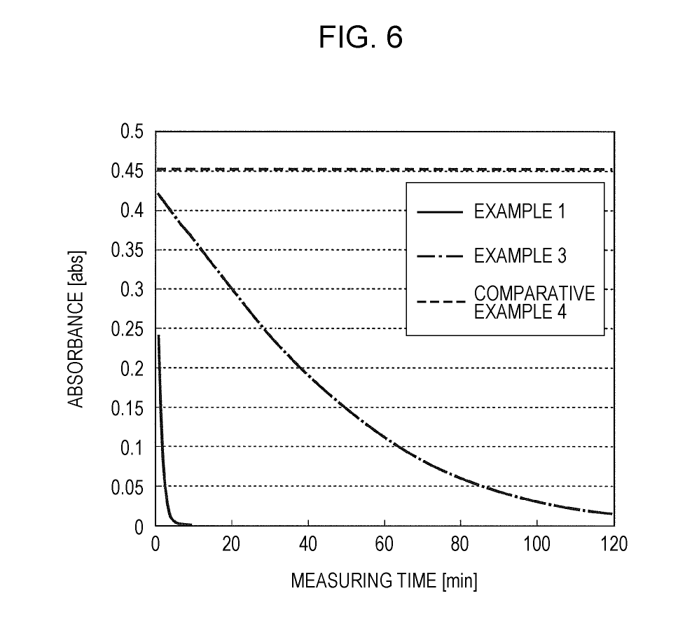

FIG. 6 shows the results of a test of indigo carmine decomposition by the liquid samples of Examples 1 and 3 and Comparative Example 4 after being left to stand for 24 hours. Herein, the liquid samples of Examples 1 and 3 and Comparative Example 4 were left to stand for 24 hours and were then brought into contact with indigo carmine.

As shown in FIG. 6, the results of Examples 1 and 3 and Comparative Example 2 were substantially the same as those shown in FIG. 5. The liquid samples of Examples 1 and 3 maintained the decomposition ability after the elapse of 24 hours. Accordingly, the plasma-treated phosphate buffer solution retained high decomposition ability regardless whether acidification was performed thereafter or not.

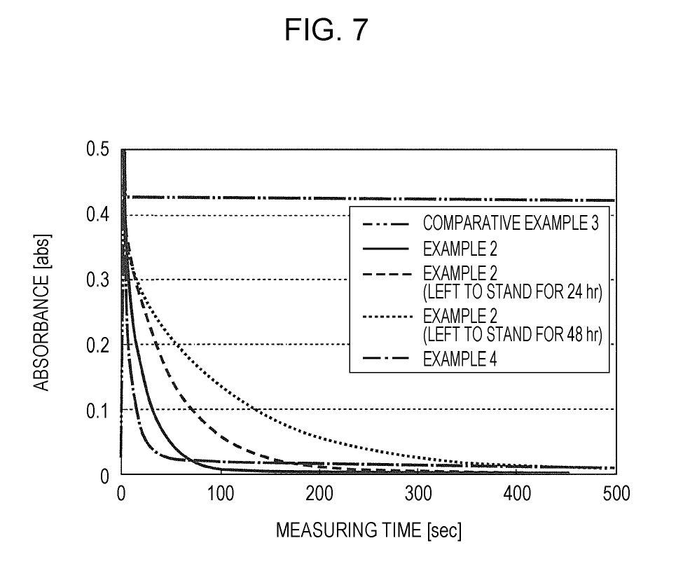

FIG. 7 shows the results of a test of indigo carmine decomposition by the liquid samples of Examples 2 and 4 and Comparative Example 3. FIG. 7 shows the results of the decomposition test immediately after the generation of liquid samples of Examples 2 and 4 and Comparative Example 3 and also the results of the decomposition test after leaving the liquid sample of Example 2 to stand for 24 hours or 48 hours.

As shown in FIG. 7, the liquid sample of Example 2 (i.e., plasma-treated alkaline aqueous sodium hydroxide solution) shew high decomposition ability. The liquid sample of Example 2 had sufficiently high decomposition ability after the elapse of 48 hours.

The liquid sample liquid sample of Example 4 (i.e., aqueous sodium hydroxide solution acidified after plasma treatment) shew high decomposition ability. The liquid sample of Example 4 shew high decomposition ability compared to the liquid sample of Example 2 not left to stand.

In contrast, the liquid sample of Comparative Example 3 (i.e., plasma-untreated aqueous sodium hydroxide solution) did not have decomposition ability. Accordingly, the comparison between Example 2 and Comparative Example 3 demonstrates that plasma treatment contributes to expression of the decomposition ability of a treatment liquid.

The results of Examples 1 to 4 demonstrate that a plasma-treated liquid has high decomposition ability, i.e., a high activity, regardless whether the liquid before the plasma treatment is an alkaline buffer solution or an alkaline aqueous solution. Accordingly, the pH of the liquid to be plasma-treated is not particularly limited, as long as the liquid is alkaline.

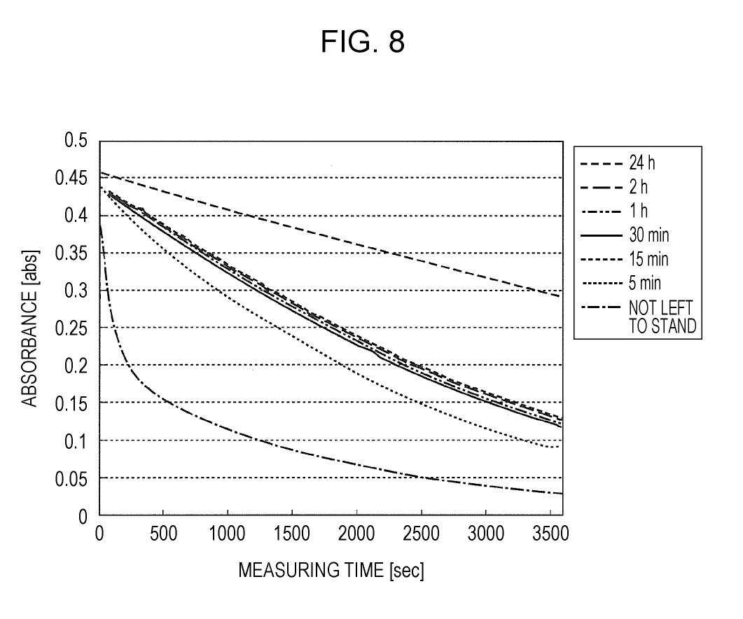

FIG. 8 shows the results of a test of indigo carmine decomposition by the liquid sample of Comparative Example 1 left to stand for predetermined periods of time. The explanatory notes in FIG. 8 show the times for which the samples were left to stand.

As shown in FIG. 8, in Comparative Example 1, the liquid sample left to stand for only 5 minutes immediately after the termination of the plasma treatment needed a longer time for decomposing indigo carmine. That is, the liquid sample (i.e., plasma-treated standard solution) of Comparative Example 1 decreased the decomposition ability within a short time. The decomposition ability of the liquid sample of Comparative Example 1 continued to decrease with the elapse of the time and highly decreased at the elapsed time of 24 hours.

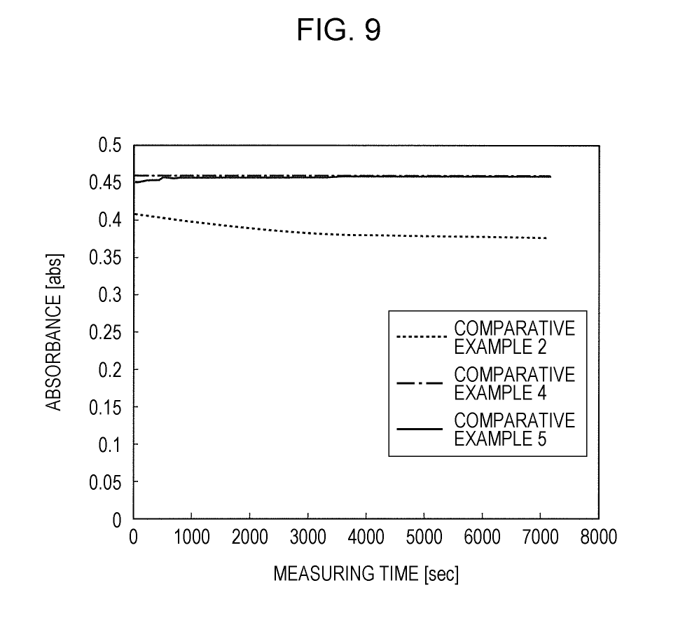

FIG. 9 shows the results of a test of indigo carmine decomposition by the treatment liquids of Comparative Examples 2, 4, and 5.

As obvious from FIG. 9, the liquid sample (i.e., acidic phosphate buffer solution) of Comparative Example 4 and the liquid sample (i.e., neutral phosphate buffer solution) of Comparative Example 5 did not substantially have decomposition ability.

The liquid sample (i.e., alkaline phosphate buffer solution) of Comparative Example 2 also did not substantially have decomposition ability. As generally known, indigo carmine partially forms a leuco structure in an alkaline solution having a pH of 11 or more to reduce the absorbance at 610 nm, which is the cause of the low initial absorbance in Comparative Example 2. The reduction in the absorbance is reversible, and the absorbance therefore returns to a value equivalent to that in Comparative Example 4 or 5 by adjusting the pH to 11 or less. However, indigo carmine is gradually decomposed when continuously mixed with an alkaline solution having a pH of 11.5 or more for a long time, resulting in a reduction in absorbance.

As obvious from Comparative Example 2, it was demonstrated that an alkaline phosphate buffer solution also did not have strong decomposition ability, unlike Examples 1 and 3.

### Modification Example

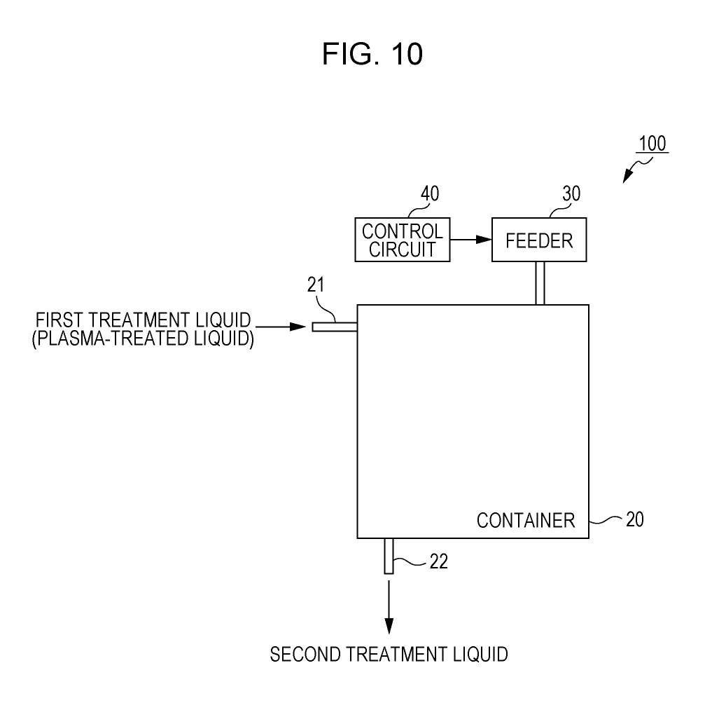

In the above-described embodiments, a structure of the treatment liquid generation apparatus 10 including a plasma generator 50 has been described, but is not limited thereto. The treatment liquid generation apparatus may not have the plasma generator 50 as in the treatment liquid generation apparatus 100 shown in FIG. 10. FIG. 10 shows the structure of the treatment liquid generation apparatus 100 according to a modification example.

As shown in FIG. 10, the treatment liquid generation apparatus 100 includes a container 20, a feeder 30, and a control circuit 40. The container 20 receives, for example, an alkaline first treatment liquid plasma-treated in advance with another apparatus, through an inlet 21. The operation of the feeder 30 and the control circuit 40 is the same as that in the above-described embodiment. However, the treatment liquid generation apparatus 100 according to this modification example does not have the plasma generator 50, the feeder 30 and the control circuit 40 may not perform the operation involved in the plasma generator 50.

The treatment liquid generation apparatus 100 according to the modification example can generate an acidic treatment liquid having a high activity as in the above-described modification example. In addition, since no plasma generator may be provided to the apparatus, decomposition and/or sterilization of an object can be performed even at a place apart from a plasma generator.

### Other Embodiments

The method of generating a treatment liquid, the treatment liquid generation apparatus according to one or more aspects have been described based on Embodiments, but the present disclosure is not limited to these Embodiments. The present disclosure also encompasses embodiments provided by applying various modifications that can be conceived by those skilled in the art to the above-described Embodiments and embodiments established by combining components in different Embodiments, without departing from the gist of the present disclosure.

For example, the treatment liquid generation apparatus in the above-described Embodiments includes a feeder 30 for supplying a pH regulator and is structured such that the control circuit 40 instructs the feeder 30 to supply a pH regulator to generate a second treatment liquid. However, in an aspect of using only a first treatment liquid, such as a structure is not necessarily required.

For example, in the above-described Embodiments, an example of a plasma generator 50 that generates plasma 92 in a liquid 90 has been described. However, the plasma generator 50 may generate plasma 92 near the liquid 90. For example, at least one of the first electrode 52 and the second electrode 53 may be disposed in a gas without being in contact with the liquid 90.

For example, when plasma 92 is generated near the surface of the liquid 90, the gas on or near the surface of the liquid 90 is exposed to the plasma 92. As a result, active species, such as ions, molecules, or radicals, are probably produced in the liquid, thereby causing first treatment liquid to be generated. In addition, nano-bubbles encapsulating the air to which the plasma has been applied can be probably generated. Furthermore, acidification of the first treatment liquid probably produces other active species in the liquid by means of the activities of the active species, such as ions, molecules, or radicals, generated by plasma treatment and the nano-bubbles. As a result, a second treatment liquid having an activity can be prepared.

In addition, in the above-described Embodiments, for example, although the pH regulator used was sulfuric acid or an aqueous sodium hydroxide solution, nitric acid or ammonia water also can be used, and any material that can change pH can be used. For example, an ordinary household detergent or lemon juice can also be used as a pH regulator.

For example, in the above-described Embodiments, the pH may be adjusted by electrolysis instead of the use of a pH regulator. For example, a container is divided into a first region and a second region by a barrier membrane, and the first region contains a plasma-treated liquid, and the second region contains a certain liquid. An electrode A is disposed in the first region, and an electrode B is disposed in the second region. In this structure, an application of a voltage between the electrode A and the electrode B electrolyzes the plasma-treated liquid. For example, in a case that a plasma-treated liquid having a pH of 9 or more is contained in the first region, the electrode A and the electrode B are used as a positive electrode and a negative electrode, respectively, and a voltage is applied such that the electrode A is positive with respect to the electrode B. As a result, the pH of the plasma-treated liquid is decreased. On this occasion, the change in pH may be monitored with, for example, the above-mentioned pH sensor.

For example, a liquid treatment apparatus may comprise: a container that contains a liquid; a plasma generator that generates plasma in or near the liquid, the plasma generator including a pair of electrodes and a power supply that applies a voltage to the pair of electrodes; and a control circuit that controls the plasma generator. The control circuit may instruct the plasma generator to: start generation of plasma, and stop the generation of plasma when the average pH per unit time of the liquid is in a predetermined range of not less than 9.

For example, a liquid treatment apparatus may comprise: a container that contains a liquid; a feeder that supplies a pH regulator to the container; and a control circuit that controls the feeder. The control circuit may instruct the feeder to supply the pH regulator to the container to change the pH of the plasma-treated liquid to less than 6, when the container contains a plasma-treated liquid having a pH of 9 or more, the plasma-treated liquid being the liquid that has been treated with plasma generated in or near the liquid.

For example, a liquid treatment apparatus may comprise: a container that contains a liquid; a pair of electrodes; a power supply that applies a voltage to the pair of electrodes; and a control circuit that controls the power supply. The control circuit may instruct the power supply to apply a voltage to the pair of electrodes to reduce the pH of the plasma-treated liquid to less than 6, when the container contains a plasma-treated liquid having a pH of 9 or more, the plasma-treated liquid being the liquid that has been treated with plasma generated in or near the liquid.

The above-described Embodiments can be subjected to a variety of, for example, modifications, replacements, additions, or omissions within the scope of the claims or a scope equivalent thereto.

The method of generating a treatment liquid and so on according to the present disclosure can generate a treatment liquid having a high activity and excellent durability of the activity. Accordingly, the method can be used in, for example, decomposition of an organic material or sterilization of microorganisms, bacteria, etc.

What is claimed is:

1. A method comprising:
preparing a liquid having a pH of 9 or more; and
generating plasma in or near the liquid to generate a plasma-treated liquid having a pH of 9 or more.

2. The method according to claim 1, further comprising:
reducing the pH of the plasma-treated liquid to less than 6.

3. The method according to claim 2, wherein
in the reducing of the pH of the plasma-treated liquid, (i) an acid or salt; (ii) a solution containing at least one of acids and salts; (iii) a gas or solid that is dissolvable in the plasma-treated liquid to show acidity; or (iv) a solution containing a microorganism producing the gas or solid is added to the plasma-treated liquid.

4. The method according to claim 2, wherein
in the reducing of the pH of the plasma-treated liquid, the plasma-treated liquid is electrolyzed.

5. The method according to claim 1, further comprising:
bringing the plasma-treated liquid having a pH of 9 or more into contact with an object to be treated.

6. The method according to claim 2, further comprising:
bringing the plasma-treated liquid having a pH of less than 6 into contact with an object to be treated.

7. The method according to claim 1, wherein
the plasma-treated liquid is generated, while being in contact with an object to be treated.
